(19) United States
(12) Patent Application Publication
(10) Pub. No.: US 2024/0164714 A1
(43) Pub. Date: May 23, 2024

(54) SMART SHOES FOR DIABETICS

(71) Applicant: KING FAISAL UNIVERSITY, AL-AHSA (SA)

(72) Inventors: AHMED SULAIMAN ALSAFRAN, AL-AHSA (SA); NOOR ALJADDOUH, AL-AHSA (SA); MAYASAM ALSHEHAB, AL-AHSA (SA); ATHEER ALROWISHED, AL-AHSA (SA); MASOUMAH ALATAFI, AL-AHSA (SA); NADA ALHUMEED, AL-AHSA (SA)

(21) Appl. No.: 17/989,319

(22) Filed: Nov. 17, 2022

(51) Int. Cl. A61B 5/00; A43B 7/00; A61B 5/0205; A61B 5/1455

(52) U.S. Cl. CPC A61B 5/6807; A43B 7/00; A61B 5/02055; A61B 5/14552; A61B 5/7267; A61B 5/7275; A61B 2560/0214; A61B 2560/0247; A61B 2562/0219; A61B 2562/0247

(57) ABSTRACT

A device, system, and method for detecting and monitoring the health condition of a patient's feet by using a foot worn sensing, data collecting, and data processing device. The foot worn device further communicates with either remotely located computers or a proximally located handheld device to provide the wearer with vital sign parameters about the wearer's diabetic condition. Additionally, the data processing includes the use of artificial intelligence methods to assimilate the vital sign parameters and arrive at a prediction about the wearer's diabetic condition.

### BACKGROUND

### 1. Field

This disclosure is directed towards providing a smart scientific solution and device to reduce or eliminate the risk of gangrene in feet and thus reduce the possibility of amputations and is provided in the form of a shoe insert.

### 2. Description of the Related Art

Global statistics show that around 536.6 million people who are diabetic patients are at risk of having diabetic feet. More particularly, the risk includes a diagnosis of gangrene in a diabetic patient's feet which occurs when blood flow to a specific tissue area of the foot is disrupted over a prolonged period of time, which can result in tissue decay and death. Additionally, when superficial ulcers appear on a foot or toe, the foot or toe also risks being amputated. Ideally, what is needed is a way to monitor certain vital signs in the foot to help the wearer and a health practitioner to monitor the health of the foot such that deleterious condition, such as gangrenous decay and tissue death can be avoided, thus leading to an improved quality of life for the diabetic patient.

CN 213604736 U discloses intelligent monitoring shoes for diabetic high-risk feet. The shoes include a sole and an upper, a plurality of temperature and humidity monitoring devices embedded in the sole and the upper, and a control module and a wireless transmission module in the sole. The temperature and humidity monitoring devices are electrically connected with the control module. The control module is electrically connected with the wireless transmission module which communicates with remote monitoring equipment. The remote monitoring equipment includes a data receiving module, a data analysis and storage module, and a wanting module where the data analysis and storage module is connected to the data receiving module and warning module, respectively. The sole is also provided with a heat dissipation insole.

CN 214179366 discloses a rehabilitation shoe that detects the blood circulation status in the feet of diabetic foot patients. The shoe incorporates a sole, a vamp, and a host, wherein the host comprises an induction detection module, an A/D conversion module, a signal comparison module, a data storage module, and, a voice and, display module. The data storage module stores induction data in a normal range. The signal detected by the induction detection module is compared with induction detection data in a normal range after the induction detection data of the target user is obtained through operation processing by the signal comparison module. The voice module and the display module are used for broadcasting or displaying the induction detection data and/or the comparison result. The rehabilitation shoe is used only for wearing when detecting the blood circulation condition of the foot of the patient and can complete the indexes such as dorsal artery pulsation, percutaneous oxygen partial pressure, ankle brachial index, foot skin temperature and the like only by simple operation without the guidance of a healthcare professional such as a doctor or nurse. The rehabilitation shoe is optimally suited for home use.

Frikart et al., (US 2007/0233206 A1) discloses a method and arrangement for monitoring a medical device which transmits messages via a communication network to a communication device, forwarding the same or another kind of message via the same or another communication network to a further communication device. The method produces a message in the medical device, transmitting the message to the communication device, and forwarding the message to another communication device such that the medical device can be remotely controlled. The method allows for the remote monitoring of an insulin pump and/or blood sugar meter and/or a continuously measuring blood sugar sensor, e.g., by the parents of the pump wearer, by professional care-givers, or by other suitable monitors (e.g., humans, computers, electromechanical devices, etc.). The wireless fully automated system, in which the transmission to the further communication appliance is initiated by the insulin pump and/or the blood sugar meter and/or the sensor, allows matters to be managed without any handling by or involvement of the pump wearer and/or user of the blood sugar meter.

There remains a need for a wearable shoe that provides a diabetic patient with the ability to closely monitor the condition of the diabetic patient's feet through robust data collection and onboard signal and data gathering and processing that further makes use of artificial intelligence technology to predict future circumstances of the diabetic patients.

### SUMMARY

The device, system, and method as discussed herein can detect and monitor certain vital signs of a diabetic patient such as temperature, humidity, heart rate, and oxygen level, such that the vital signs data can be stored, analyzed, communicated remotely, and used to predict future circumstances, such as the possible occurrence of foot gangrene and superficial ulcers in a diabetic patient's feet, through the use of artificial intelligence technology. The storage, analysis and prediction take place in the device, which is resident on or about a diabetic patient's foot, occasionally herein referred to as a Smart Shoe, Smart Shoe device, shoe insert, shoe insert device. Smart Shoe insert, or Smart Shoe insert device.

In one embodiment, the present subject matter relates to a device for detecting vital sign parameters about a diabetic patient's foot, the device comprising a shoe insert comprising one or more of: a) a topmost layer of a sole insert; b) a middle layer of the sole insert; c) a bottom layer of the sole insert; d) a control module mounted on the middle layer of the sole insert; e) a plurality of pressure sensors located above the bottom layer of the sole insert which, when downwardly pressed due to a pressure provided by the diabetic patient's foot, provide a plurality of corresponding feedback signals to the control module to indicate that the shoe is being worn by said diabetic patient; f) a temperature sensor for measuring a temperature of said foot; g) a humidity sensor for measuring a humidity of said foot; h) a blood oxygen sensor for measuring an oxygen concentration of the blood in said foot; i) a heart-rate sensor for measuring a heart-rate in said foot; j) a wireless charging antenna located between said middle layer and said bottom layer of said sole insert, said wireless charging antenna providing charging power for said device; and k) a battery mounted atop said control module for storing standby power to said device.

In some embodiments, the control module can further comprise a microcontroller configured for implementing a method for collecting data, analyzing the collected data, determining a condition classification, and generating a prediction of future diabetic health circumstances; a wireless communication module configured for wirelessly communicating to remote equipment or to a proximal handheld device; an antenna connected to the wireless communication module wherein the antenna is configured to transmit a wireless communication to said remote equipment or to said proximal handheld device; and an accelerator. In certain embodiments, the microcontroller can be configured for wireless charging. In this regard, the wireless charging antenna can provide transmitted charged power to all components of the device. In use, the wireless charging antenna can operate in the 130 KHz-140 KHz range and can have a gap of 50 mm.

In one embodiment, the battery used in the present device is a Lithium Polymer battery with over 48 hours of Bluetooth® standby time. In another embodiment, the battery operates at 50 mAH.

In certain embodiments, the plurality of pressure sensors can have an arrangement of two pressure sensors in a heel area of said device and six pressure sensors in an area in front of where an arch of said foot is located on the device, in use.

In further embodiments, certain sensors can be integral to a single component. By way of non-limiting example, the blood oxygen sensor and the heart rate sensor can be integral to one component.

In additional embodiments of the present subject matter, the determining a condition classification uses artificial intelligence to determine the condition classification, wherein said artificial intelligence comprises a neural network model with linear regression, model training, and inference. In other additional embodiments, the generating a future prediction of diabetic health circumstances uses artificial intelligence to generate the future prediction, wherein said artificial intelligence generates the future condition based on current data collection and past data collection, recurrent neural network training, and recurrent neural network inference.

These and other features of the present subject matter will become readily apparent upon further review of the following specification.

### BRIEF DESCRIPTION OF THE DRAWINGS

FIG. 1 is an exploded view of the Smart Shoe insert.

FIG. 2 is a diagram of the various sensors in the Smart Shoe device.

FIG. 3 is a circuit diagram of the temperature sensor used in the Smart Shoe device.

FIG. 4 is a circuit diagram of the humidity sensor used in the Smart Shoe device.

FIG. 5 is a circuit diagram of the heartbeat sensor and the blood oxygen level sensor used in the Smart Shoe device.

FIG. 6 depicts where on the feet the signals are measured.

FIG. 7 is a list of potential components used in the Smart Shoe device.

FIGS. 8A-8C are CAD diagrams of the Smart Shoe device.

FIG. 9 is a decision tree flowchart demonstrating how the AI in the Smart Shoe device makes its determination and generates future predictions.

FIG. 10 is a decision tree flowchart of the Smart Shoe device which determines condition classification of the diabetic patient's health.

Similar reference characters denote corresponding features consistently throughout the attached drawings.

### DETAILED DESCRIPTION OF THE PREFERRED EMBODIMENTS

The Smart Shoe device of this disclosure provides a construction and arrangement ensuring that diabetic patients who use the device can readily ascertain the medical condition of their diabetic feet and further help accurately predict any possible medical complications which may arise based on a deteriorating condition of their feet. In one embodiment, the present device is intended for use by a patient, such as a diabetic patient, at home in order to self-monitor their medical condition. However, as will be apparent, the Smart Shoe device of this disclosure is also intended to work in conjunction with one or more remote health-care professionals, by way of non-limiting example, doctors, nurses, physician assistants, and the like, particular in instances of virtual medical appointments.

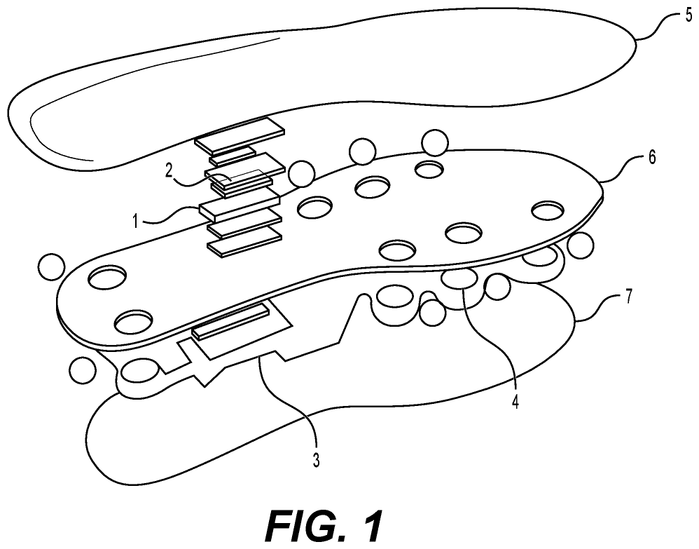

The Smart Shoe device is constructed as a shoe insert with three layers as shown in FIG. 1, namely a top layer (5), a middle layer (6), and a bottom layer (7). Accordingly, the present device may be herein referred to as a Smart Shoe, Smart Shoe device, shoe insert, shoe insert device, Smart Shoe insert, or Smart Shoe insert device, each of which phrases may be used inter changeably herein.

Most of the electronic components in the device are located either in the middle layer (6) or the bottom layer (7) of the shoe insert. The middle layer of the shoe insert has eight holes, two in the heel, and six in front of the arch portion of the insert, or in an area in front of an where an arch of said foot is located on the device, in use, in which eight pressure sensors reside (labeled by numbered discs). The eight pressure sensors provide bio-feedback to the control module (1) that a diabetic patient is wearing the Smart Shoe device when they are downwardly pressed due to a pressure provided by the diabetic patient's foot.

Also residing in the middle layer (6), the control module (1) can include a TI MSP430® microcontroller. However, any suitable microcontroller can be used. In addition, the device can include a CSR BC04-EXT Bluetooth® Flash Module for Bluetooth® enabled communication. The Smart Shoe device can communicate with remote equipment as described below or with a synced phone or tablet which has an app that works in conjunction with sensor data measured with the Smart Shoe to help the patient monitor their health condition and take action accordingly. The TI MSP430® microcontroller also has wireless charging capabilities.

In additional embodiments, the control module (1) can further comprise a Bluetooth® antenna, such as, by way of non-limiting example, the Nordic NRF51822, which is a general purpose, ultra-low power SoC (System on Chip) antenna ideally suited for Bluetooth Low Energy and 2.4 GHz proprietary wireless applications, and is supported with 2.4 GHz proprietary, such as Gazell. In other embodiments, the control module (1) can further comprise one or more of a Flash data storage unit, such as, by way of non-limiting example, a 32 MB Flash unit for data storage; and an accelerator, such as, by way of non-limiting example, a three axis linear accelerator, for example, a LIS3DH ultra-low-power high-performance three-axis linear accelerometer belonging to the “nano” family, with digital I2C/SPI serial interface standard output. The device features ultra-low-power operational modes that allow advanced power saving and smart embedded functions. The LIS3DH has dynamically user-selectable full scales of ±2 g/±4 g/±8 g/±16 g and is capable of measuring accelerations with output data rates from 1 Hz to 5.3 kHz. The self-test capability allows the user to check the functioning of the sensor in the final application. The device may be configured to generate interrupt signals using two independent inertial wake-up/free-fall events as well as by the position of the device itself. Thresholds and timing of interrupt generators are programmable by the end user on the fly. The LIS3DH has an integrated 32-level first-in, first-out (FIFO) buffer allowing the user to store data in order to limit intervention by the host processor.

As shown in FIG. 1, the Smart Shoe can also include a battery (2) for providing power to all the components of the shoe insert device, such as the control module (1), the wireless charging antenna (3), and the digital pressure sensors (4). One non-limiting example of a battery (2) useful herein is a Lithium polymer (Lipo) battery, which, compared with other batteries, has the characteristics of high energy, miniaturization, and is more lightweight. For the ultrathin characteristics, it can be made into batteries of different shapes and capabilities to meet the needs a particular product. The theoretical minimum thickness of a Lipo battery can reach 0.4 mm.

In certain embodiments, the Lithium polymer battery can comprise several identical parallel secondary cells to increase the discharge current, or several battery packs in series to increase the available voltage. The Lipo battery in certain embodiments provides for 48 hours of Bluetooth® standby time. Based upon the sizing flexibility of the Lipo battery, it's use as a power source is optimal because of having to be placed into a cavity of the present shoe insert.

In other embodiments, the Smart Shoe insert can also have a wireless charging antenna (3) between the middle layer (6) and the bottom layer (7). The wireless charging antenna can operate in the 130 KHz-140 KHz range, can have a 50 mm gap, and can function to provide transmitted power to the rest of the shoe insert.

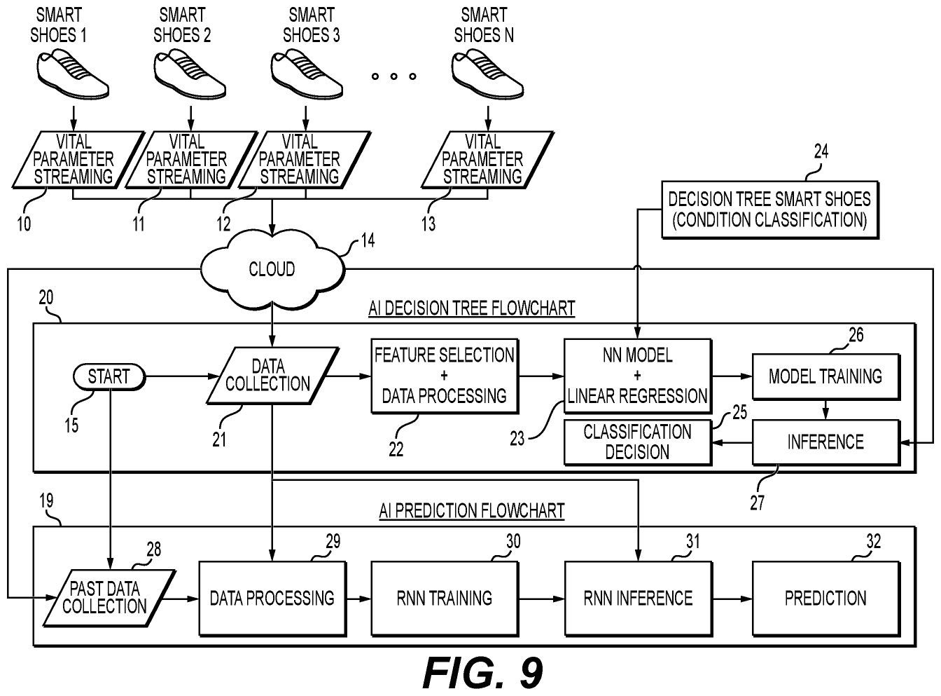

In some embodiments, the control module (1) implements methods for monitoring the health of a diabetic foot and uses artificial intelligence (AI) modality to predict future circumstances of a patient's foot health and overall diabetic health. In FIG. 9, a cloud-based implementation of the method is shown. Individual patients each having a Smart Shoe (Smart Shoe 1 . . . Smart Shoe N) have their vital parameters streamed (10, 11, 12, 13) to the cloud by each control module on each individual smart shoe insert, these vital parameters including but not restricted to: temperature, humidity, heartbeat, pulse, and blood oxygen level. From the cloud (14), streamed vital parameters are collected as data and sent to two parallel artificial intelligence processes as implemented on remotely located computers or other processing devices. The first process is an AI Decision Tree Flowchart (20), which, after receiving a start command (15), executes a step of current data collection (21) from the cloud (14), and performs a feature selection and additional data processing step (22) on the output of the data collection step.

The AI Decision Tree Flowchart (2) further shows a neural network modelling step using linear regression (23) and a Smart Shoe decision tree (24) for conditions classified as a first input and the output of the feature selection and data processing step (22) as a second input, a model training step (26) based upon the output of the neural network modelling step (23), an inference step (27) with inputs from the model training step output (26) and a cloud based input (14) of vital streaming parameters (10, 11, 12, 13), and lastly a classification decision step for determining a current condition classification of the patients' diabetic foot health (25) based upon the inference step output. One advantage of regression analysis is to provide an understanding of the strength of relationships between the measured variables of concern—in this case, temperature, humidity, blood oxygen levels, heartbeat, and pulse. Regression analysis further indicates much of the total variability in the data is explained by the model. Furthermore, regression analysis indicates what predictors in a model are statistically significant and which are not. Regression analysis provides a more robust understanding of statistical inference overall.

As also shown in FIG. 9, the AI Prediction Flowchart (19) starts concurrently with the AI Decision Tree Flowchart (20) and includes the step of past data collection (28) based upon its own start command (15) and a data collection of past data collection from the cloud (14), a data processing step (29) based upon the past and current collected data (21, 28), a recurrent neural network training step (30) operative upon the output of the data processing step (29), a recurrent neural network (RNN) inference step (31) based upon the recurrent neutral network training step output (30) and the current data collection step (21), and finally, a prediction step (32) based upon the output of the recurrent neural network inference step (31). The RNN works on the principle of saving the output of a particular layer and feeding this back to the input in order to predict the output of the layer. The advantages of a RNN is that a RNN can handle sequential data, accepting the current input data, and previously received inputs. RNNs can memorize previous inputs due to their internal memory. Preferably, a many to one RNN is used. This RNN takes a sequence of inputs and generates a single output. For instance, taking the vital parameters as a sequence of inputs and generating a single output, i.e., a prediction.

Recurrent Neural Networks enable time-dependent and sequential data problems to be modeled. However, RNNs can be hard to train due to the problem of vanishing gradients. The gradients carry information used in the RNN, and when the gradient becomes too small, the parameter updates become insignificant. This makes the learning of long data sequences difficult. While training a neural network, if the slope tends to grow exponentially instead of decaying, this is called an Exploding Gradient. This problem arises when large error gradients accumulate, resulting in exceptionally large updates to the neural network model weights during the training process. Long training time, poor performance, and bad accuracy are the significant issues in gradient problems. A popular and efficient way to deal with gradient problems is the use of Long Short-Term Memory Networks (LSTMs). LSTMs are a special kind of RNN-capable of learning long-term dependencies by remembering information for extended periods is the default behavior.

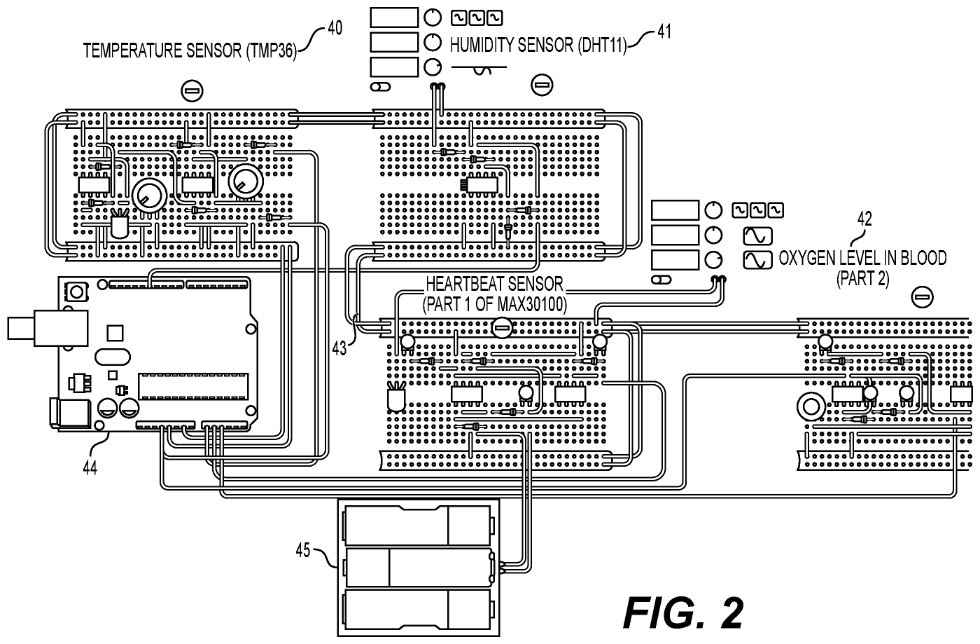

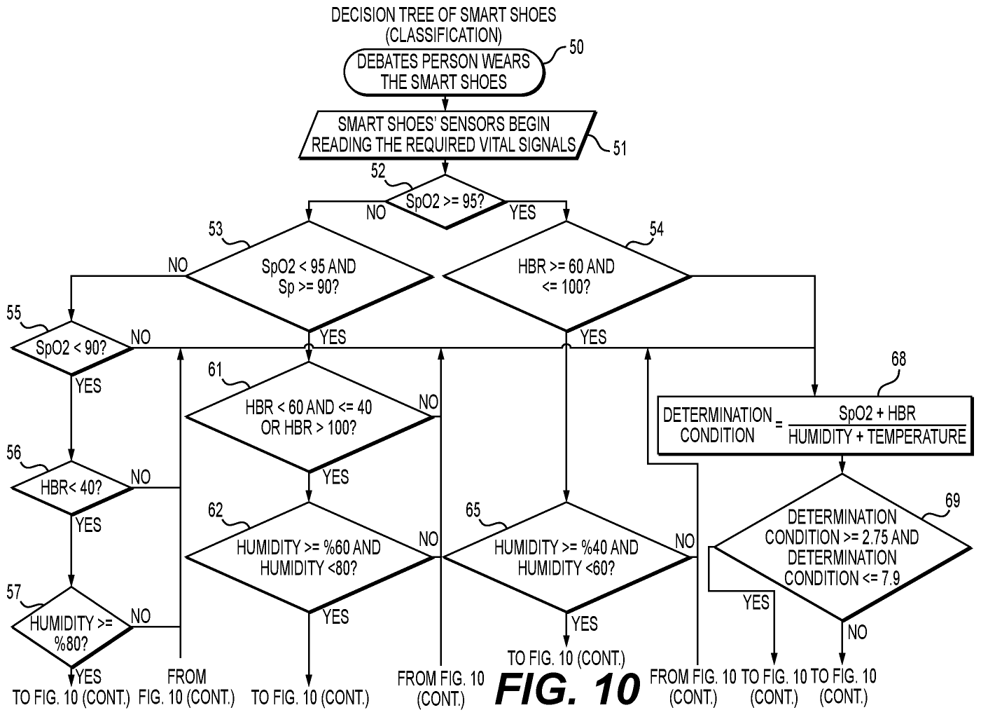

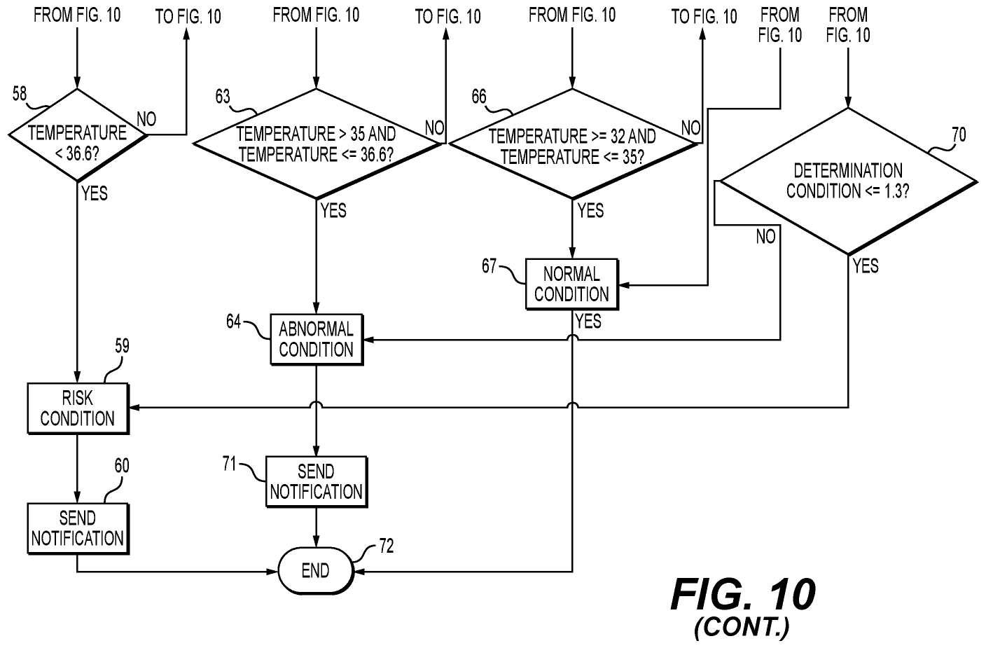

FIG. 10 depicts the Decision Tree Flowchart for Condition Classification which is implemented in the AI Decision Tree Flowchart (FIG. 9, element 24) and provides an input into the Neural Network Model With Linear Regression (FIG. 9, element 23). As shown in FIG. 10, the process starts by detecting whether the Smart Shoe device is being worn (50) by reading the pressure sensors in the Smart Shoe device (FIG. 1, elements 4). Once that determination in FIG. 10 is affirmed, the data from sensors in FIG. 2 such as the temperature sensor (40), humidity sensor (41), pulse oximeter sensor (42), and heartbeat sensors are read out (52) and stored for analysis (FIG. 1, control module 1).

In FIG. 10, SpO2 stands for the concentration of oxygen level in the blood, and HBR stands for the heartbeat rate of the wearer. The output from the sensor reading step (51) is input into a first determination step (52) which determines if SpO2≥95. The NO condition of determination step (52) leads to a further determination step (53) which evaluates whether 90≤SpO2<95. The YES condition of this determination step (53) is input into a chain of determination steps (61), (62), and (63), and can lead to a decision step of an abnormal condition (64) if the respective outputs from steps (61), (62), and (63) are all YES. A positive output of the abnormal condition decision step (64) generates a notification to send (71) to the wearer or to a healthcare professional in a manner as already discussed above.

The determination step (61) evaluates the heartbeat rate by reading for one of two conditions: a) 40≤HBR<60 or b) HBR>100. The determination step (62) evaluates the humidity by reading whether 60≤Humidity<80. The determination step (63) evaluates the temperature by reading whether 35° C.<Temperature≤36.67° C. A NO output at any of the determination steps (61), (62), and (63) leads that respective NO output being input into a Determination Condition step (68) for further evaluation. The NO condition of this determination step (53) is input into a chain of determination steps (55), (56), (57), and (58), and can lead to a decision step of a risk condition (59) if the respective outputs from steps (55), (56), (57), and (58) are all YES. A positive output of the risk condition decision step (59) generates a notification to send (60) to the wearer or to a healthcare professional in a manner as already discussed above.

The determination step (55) determines whether SpO2<90. The determination step (56) determines heartbeat rate by evaluating whether HBR<40. The determination step (57) evaluates the humidity by reading whether Humidity≥80. The determination step (58) determines the temperature by evaluating whether Temperature<36.6° C. A NO output at any of the determination steps (55), (56), (57), and (58) also leads that respective NO output being input into a Determination Condition step (68) for further evaluation. The YES condition of determination step (52) leads to a further determination step (54) of whether 60≤HBR≤100. The YES condition of this determination step (54) is input into a chain of determination steps (65), (66), and can lead to a decision step of a normal condition (64) if the respective outputs from steps (65), (66), are all YES. The determination step (65) evaluates the humidity rate by reading whether 40≤Humidity≤60. The determination step (66) evaluates the temperature by reading whether 32° C.<Temperature≤35° C. A NO output at any of the determination steps (65), (66) leads that respective NO output being input into a Determination Condition step (68) for further evaluation.

The Determination Condition step calculates a Determination Condition (DC) factor, where DC=(SpO2×HBR)/(Humidity×Temperature) and outputs DC to a chain of Determination Condition factor evaluation steps (69), (70). A chain of DC factor evaluation steps (69), (70) can lead to a decision step of a risk condition (59) if the respective outputs from steps (69), (70), are NO and YES. The DC factor evaluation step (69) and can lead to a decision step of a normal condition (67) if the output from step (69) is YES. The DC factor evaluation step (70) can lead to a decision step of an abnormal condition (64) if the output from step (70) is NO. The DC factor evaluation step (69) evaluates the DC by determining whether 2.75≤DC≤7.9. The DC factor evaluation step (70) evaluates the DC by determining whether 1.3≤DC. While FIG. 10 shows no send notification step after the determination of a normal condition, it is contemplated that such a feature could be added after the normal condition decision step (67) and before the end step (72) in order to provide the user an indication of the user's normal condition.

FIG. 2 shows the layout of various sensors and their connectivity to the microcontroller board (44). In certain embodiments, the microcontroller board (44) can be an Arduino Uno R3, which supports the TI MSP430® microcontroller. In this embodiment, the microcontroller board (44) has 14 digital input/output pins (of which 6 can be used as PWM outputs), 6 analog inputs, a 16 MHz ceramic resonator (CSTCE16M0V53-R0), a USB connection, a power jack, an ICSP header and a reset button. It contains everything needed to support the microcontroller and can simply connect the microcontroller to a computer with a USB cable.

The microcontroller board (44) can be powered via the USB connection or with an external power supply. In one embodiment, the power source is selected automatically. External (non-USB) power can come either from an AC-to-DC adapter or battery. The adapter can be connected by plugging a 2.1 mm center-positive plug into the board's power jack.

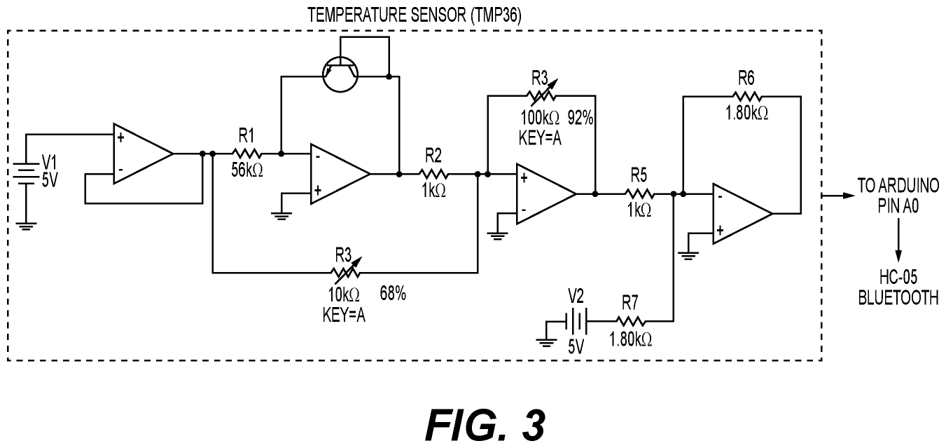

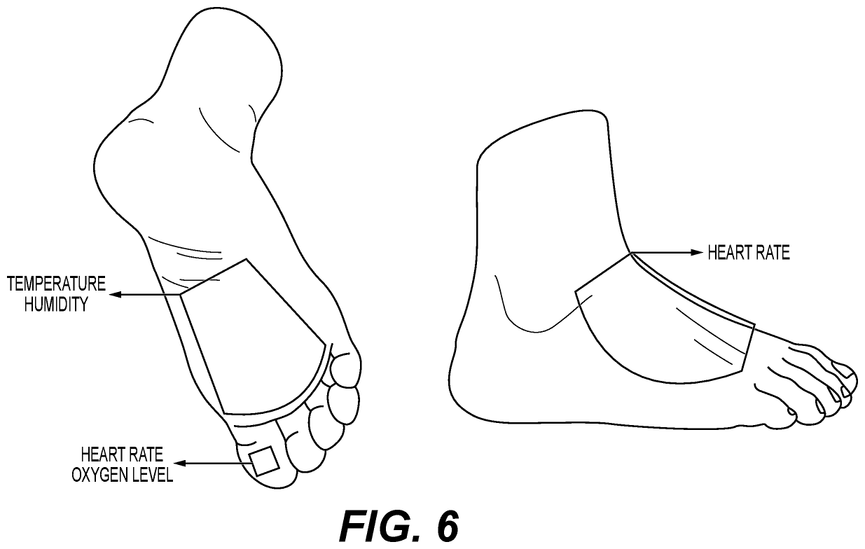

A first input to the microcontroller board (44) is the TMP36 temperature sensor (40) which is a low voltage, precision centigrade temperature sensor. It provides a voltage output that is linearly proportional to the measured Celsius temperature. It also does not require any external calibration to provide typical accuracies of ±1° C. at +25° C. and ±2° C. throughout the −40° C. to +125° C. temperature range. The output voltage can be converted to temperature easily using the scale factor of 10 mV/° C. The temperature sensor (40) can be placed in the Smart Shoe insert such that the sensor makes contact with the bottom part of the metatarsal and tarsal area of the soles of the feet as shown in FIG. 6. FIG. 3 is a detailed circuit diagram of the TMP36 temperature sensor.

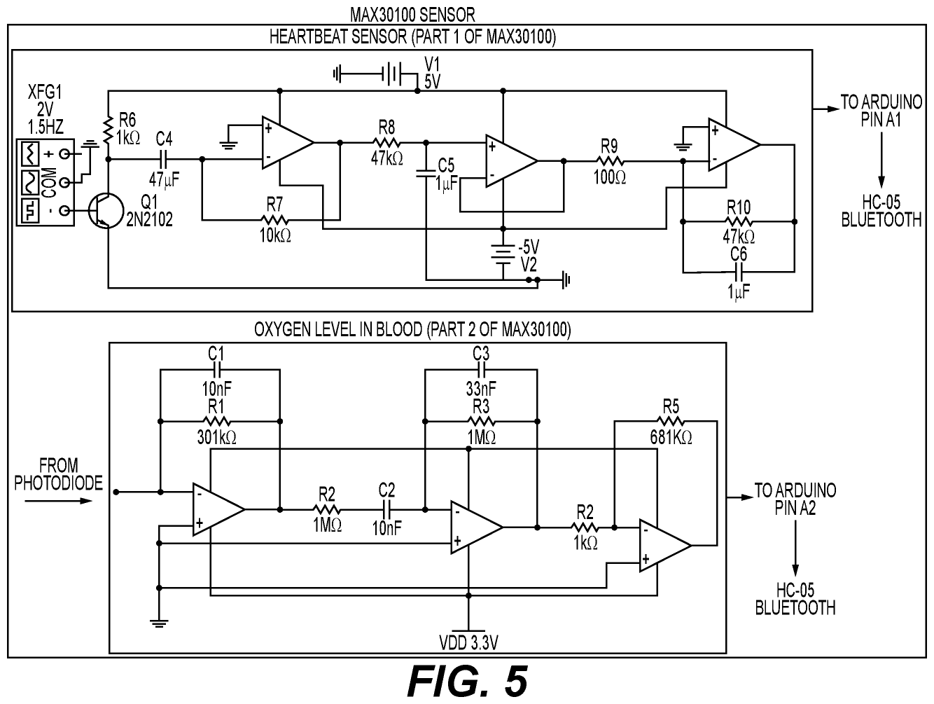

Also, FIG. 2 shows the heartbeat sensor and the blood oxygen level sensor, both resident aboard the MAX30100 according to this embodiment. The MAX30100 is an integrated pulse oximetry and heart-rate monitor sensor. It combines two LEDs, a photodetector, optimized optics, and low-noise analog signal processing to detect pulse oximetry and heart-rate signals. The MAX30100 operates from 1.8V and 3.3V power sup-plies and can be powered down through software with negligible standby current, permitting the power supply to remain connected at all times. The MAX30100 has integrated LEDs, a photo sensor, and a high-performance analog front end. Additionally, its ultra-low power operation increases battery life for wearable devices such as the present Smart Shoe insert. It also has a programmable sample rate, LED current for power savings, and an ultra-low shutdown current. The MAX30100 also provides a high SNR which provides robust motion artifact resilience, integrated ambient light cancelation, high sample rate capabilities, and fast data output capabilities. The MAX30100 is placed in the shoe insert such that it makes contact with the underside of the big toe as shown in FIG. 6. FIG. 5 is a detailed circuit diagram of the MAX30100 sensor.

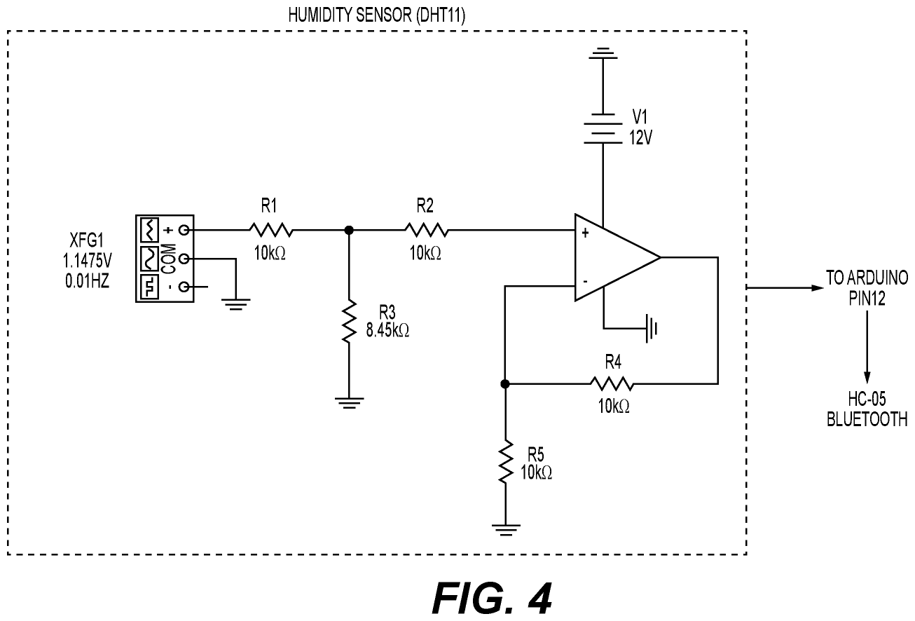

Lastly, the DHT11 is a basic, ultra-low-cost digital temperature and humidity sensor which can be employed in one embodiment herein. It uses a capacitive humidity sensor and a thermistor to measure the surrounding air, and outputs a digital signal on the data pin (no analog input pins needed). It provides new data once every 2 seconds and comes with a 4.7K or 10K resistor, can be used as a pullup from the data pin to V_{CC}. It operates on a 3V to 5V power and I/O range. It also operates with a 2.5 mA max current use during conversion and is generally dependable for 20-80% humidity readings with 5% accuracy. No more than 1 Hz sampling rate (once every second). FIG. 4 shows a detailed circuit diagram of the DHT11 humidity sensor.

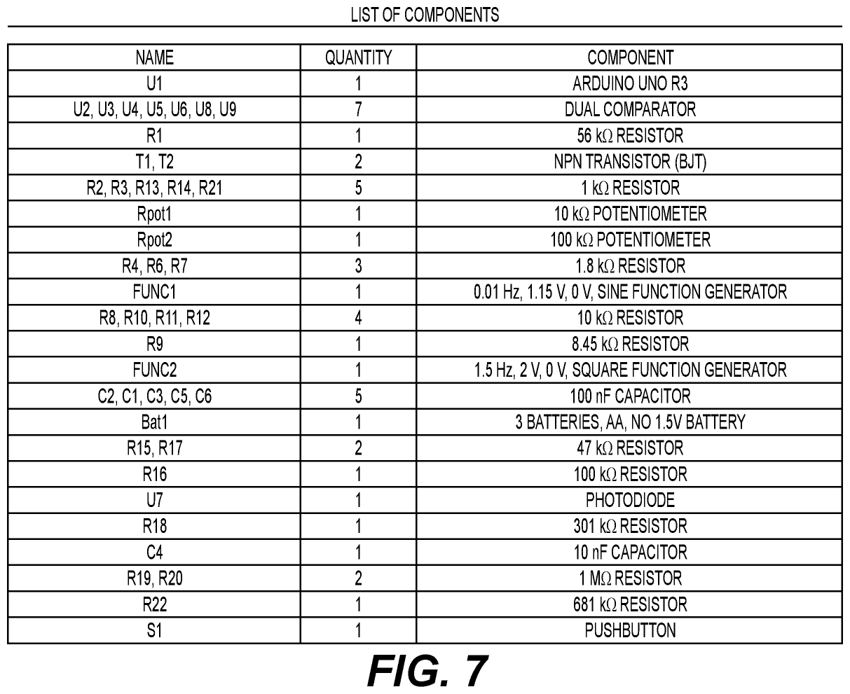

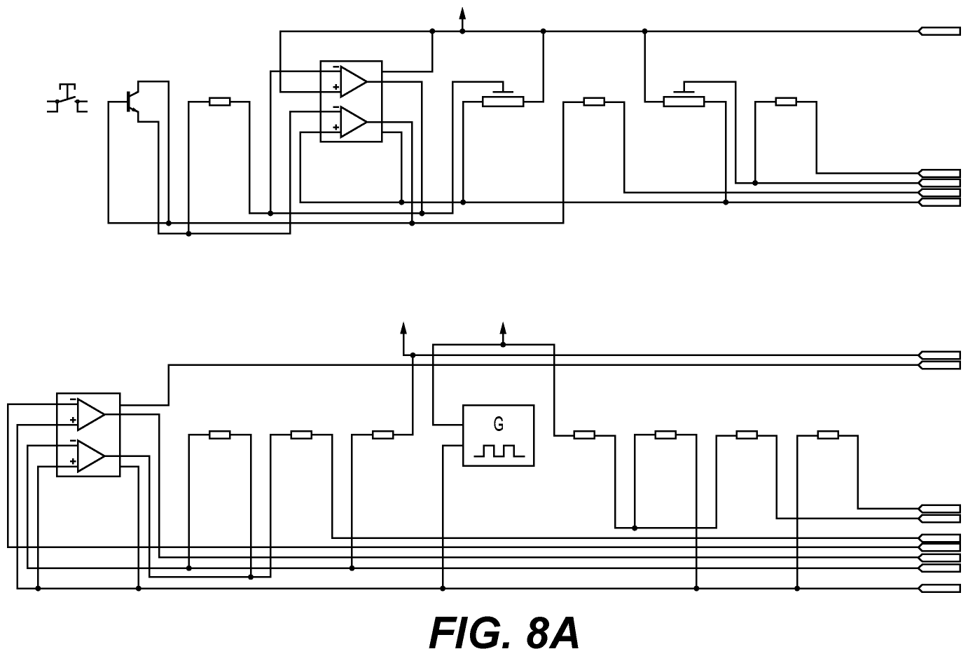

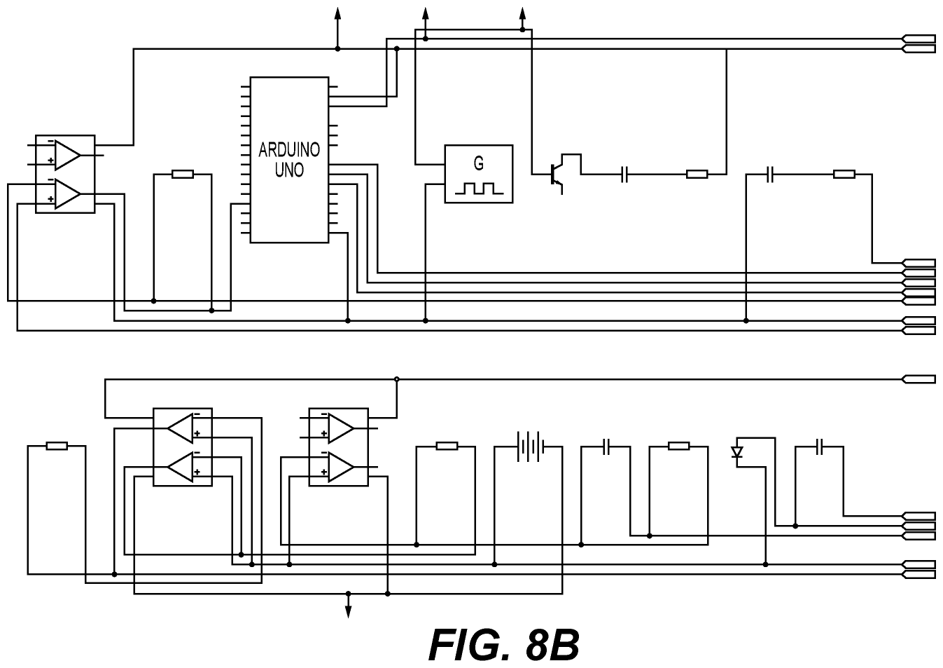

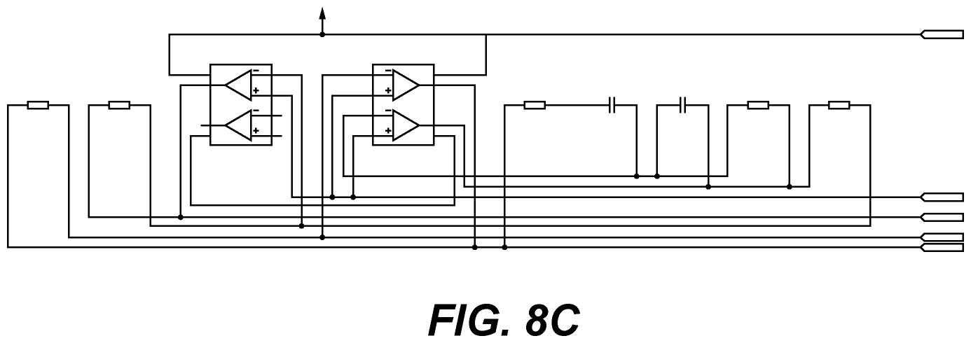

FIG. 7 is a list of components used for circuitry of the present Smart Shoe insert and FIGS. 8A-8C are CAD figures of the Smart Shoe insert which show the various connections, and layout of capacitors, resistors, potentiometers, NPN transistors, comparators, photodiodes, power sources, and wave function generators that constitute the device.

It is to be understood that the present device, system, and method for diabetic feet is not limited to the specific embodiments described above but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A device for detecting vital sign parameters about a diabetic patient's foot, the device comprising a shoe insert comprising:
a) a topmost layer of a sole insert;
b) a middle layer of the sole insert;
c) a bottom layer of the sole insert;
d) a control module mounted on the middle layer of the sole insert;
e) a plurality of pressure sensors located above the bottom layer of the sole insert which, when downwardly pressed due to a pressure provided by the diabetic patient's foot, provide a plurality of corresponding feedback signals to the control module to indicate that a shoe is being worn by said diabetic patient;
f) a temperature sensor for measuring a temperature of said foot;
g) a humidity sensor for measuring a humidity of said foot;
h) a blood oxygen sensor for measuring an oxygen concentration of the blood in said foot;
i) a heart-rate sensor for measuring a heart-rate in said foot;
j) a wireless charging antenna located between said middle layer and said bottom layer of said sole insert, said wireless charging antenna providing charging power for said device; and
k) a battery mounted atop said control module for storing standby power to said device, wherein said battery further comprises identical parallel secondary cells to increase a discharge current;
wherein said control module further comprises:
a microcontroller configured for implementing a method for collecting data, analyzing the collected data, determining a condition classification, and generating a prediction of future diabetic health circumstances, wherein said microcontroller also has wireless charging abilities for providing charging power to said device;
a wireless communication module configured for wirelessly communicating to remote equipment or to a proximal handheld device;
a communication antenna connected to the wireless communication module wherein the communication antenna is configured to transmit a wireless communication to said remote equipment or to said proximal handheld device;
an accelerometer.

2. The device for detecting vital sign parameters about a diabetic patient's foot, as recited in claim 1, wherein said battery is a Lithium Polymer battery with over 48 hours of standby time.

3. The device for detecting vital sign parameters about a diabetic patient's foot as recited in claim 2, wherein said battery operates at 50 mAH.

4. (canceled)

5. The device for detecting vital sign parameters about a diabetic patient's foot as recited in claim 1, wherein said wireless charging antenna provides transmitted charged power to all components of the device.

6. The device for detecting vital sign parameters about a diabetic patient's foot as recited in claim 5, wherein said wireless charging antenna operates in the 130 KHz-140 KHz range.

7. The device for detecting vital sign parameters about a diabetic patient's foot as recited in claim 6, wherein said wireless charging antenna has a gap of 50 mm.

8. The device for detecting vital sign parameters about a diabetic patient's foot as recited in claim 1, wherein said plurality of pressure sensors has an arrangement of two pressure sensors in a heel area of said device and six pressure sensors in an area in front of where an arch of said foot is configured to be located on the device, in use.

9. The device for detecting vital sign parameters about a diabetic patient's foot as recited in claim 1, wherein said blood oxygen sensor and said heart rate sensor are integral to one component.

10. The device for detecting vital sign parameters about a diabetic patient's foot as recited in claim 1, wherein
said determining a condition classification uses artificial intelligence to determine the condition classification, wherein said artificial intelligence comprises a neural network model with linear regression, model training, and inference; and
said generating a future prediction of diabetic health circumstances uses artificial intelligence to generate the future prediction of diabetic health circumstances, wherein said artificial intelligence generates the future prediction of diabetic health circumstances based on current data collection and past data collection, recurrent neural network training, and recurrent neural network inference.

11. A method for detecting vital sign parameters about a diabetic patient's foot, the method comprising inserting the device of claim 1 into a shoe of a patient having diabetes and measuring various vital sign parameters from the foot of the patient having diabetes using the device.

12. A method for reducing the risk of gangrene in a diabetic patient's foot, the method comprising:
inserting the device of claim 1 into a shoe of a patient having diabetes;
measuring vital sign parameters from the foot of the patient having diabetes using the device;
analyzing the measured vital sign parameters;
determining a condition classification; and
informing the patient having diabetes of their increased risk of developing gangrene in the foot in real time based on the condition classification.

13. The method according to claim 12, wherein the analyzing is conducted using artificial intelligence (AI).
